(19) United States
(12) Patent Application Publication
(10) Pub. No.: US 2026/0144672 A1
(43) Pub. Date: May 28, 2026

(54) CARRIER USED IN COLD-HOT COMPRESS DEVICE AND COLD-HOT COMPRESS DEVICE

(71) Applicant: Xiamen Ubest Electronic Technology Co., Ltd., Xiamen (CN)

(72) Inventor: Zonghu Zhu, Xiamen (CN)

(21) Appl. No.: 19/398,795

(22) Filed: Nov. 24, 2025

Related U.S. Application Data
(63) Continuation-in-part of application No. 19/015,615, filed on Jan. 9, 2025.

(30) Foreign Application Priority Data
Nov. 27, 2024 (CN) 202422904505.2
Dec. 26, 2024 (CN) 202423234976.3
Oct. 11, 2025 (CN) 202522150590.2

(51) Int. Cl. A61F 7/02; A61F 7/00

(52) U.S. Cl. CPC A61F 7/02; A61F 2007/0069; A61F 2007/0222; A61F 2007/0244

(57) ABSTRACT

A carrier used in a cold-hot compress device is configured to be connected to a main unit of the cold-hot compress device so as to receive a fluid output by the main unit, and the carrier includes a main body configured to be used for integrally wrapping one or more legs, a waist, an arm, an upper body, or a whole body of a human body.

### RELATED APPLICATIONS

This application is a continuation-in-part of U.S. patent application Ser. No. 19/015,615, filed on Jan. 9, 2025, and claims priority to Chinese patent application 202423234976.3, filed on Dec. 26, 2024, Chinese patent application 202522150590.2, filed on Oct. 11, 2025, and Chinese patent application 202422904505.2, filed on Nov. 27, 2024. U.S. patent application Ser. No. 19/015,615, Chinese patent application 202423234976.3, Chinese patent application 202522150590.2, and Chinese patent application 202422904505.2 are incorporated herein by reference.

### FIELD OF THE DISCLOSURE

The present disclosure relates to the field of nursing equipment, and in particular relates to a cold-hot compress device.

### BACKGROUND OF THE DISCLOSURE

Cold-hot compress devices usually transfer temperature to a carrier through components capable of cooling and heating, so that cold-hot compress can be applied to a nursing part attached to the carrier to achieve an effect of treatment and nursing. In the design of the existing cold-hot compress devices, semiconductors are commonly used for cooling and heating, but the semiconductors are mostly applied to small-sized cold-hot compress devices and cannot meet requirements of rapid cooling and heating when large-area cold-hot compress is required. Even if multiple groups of semiconductors are combined to form a large-area working surface, structures of the semiconductors have limitations, cannot be bent arbitrarily, and cannot meet a requirement of being close fitting with the nursing part.

### BRIEF SUMMARY OF THE DISCLOSURE

A main technical problem to be solved by the present disclosure is to provide a cold-hot compress device.

In order to solve the above technical problems, the present disclosure provides a carrier used in a cold-hot compress device. The carrier is configured to be connected to a main unit of the cold-hot compress device so as to receive a fluid output by the main unit, and the carrier comprises a main body configured to be used for integrally wrapping one or more legs, a waist, an arm, an upper body, or a whole body of a human body.

In a preferred embodiment, the fluid comprises air and liquid.

In a preferred embodiment, the main body comprises an outer layer, an inflatable layer, and a working layer. The inflatable layer and the working layer are disposed on a side of the outer layer facing skin of the human body, and the inflatable layer is configured to receive the air output by the main unit. The working layer is configured to receive the liquid output by the main unit.

In a preferred embodiment, the inflatable layer and the working layer are stacked along a direction perpendicular to the skin of the human body.

In a preferred embodiment, the fluid is a liquid, and the main body comprises an outer layer and a working layer. The working layer is disposed on a side of the outer layer facing skin of the human body, and the working layer is configured to receive the liquid output by the main unit.

In a preferred embodiment, fabrics of the working layer are divided into one or more cloth base layers and one or more thermoplastic polyurethane (TPU) layers, and the one or more TPU layers in an upper fabric and a lower fabric of the working layer are connected at a certain distance by pressing along an extending direction of the working layer.

In a preferred embodiment, the main body comprises one or more cavities that integrally wrap the one or more legs, the waist, the arm, the upper body, or the whole body of the human body. A shape of the one or more cavities is set according to a shape of a part of the human body being wrapped.

In a preferred embodiment, the main body comprises a liquid inlet and a liquid outlet in communication with the working layer.

In a preferred embodiment, a first indentation is provided in the working layer. The first indentation is configured to define a flow channel in the working layer that connects the liquid inlet to the liquid outlet, and the flow channel is disposed in a winding arrangement.

In a preferred embodiment, a second indentation is further provided in the working layer, and a certain distance is formed between the second indentation and the first indentation at a position where the second indentation is located, so as to divide the liquid flowing through the second indentation into two parts.

In a preferred embodiment, the first indentation and the second indentation are curved pressing lines.

In a preferred embodiment, the working layer is provided with press-fit points in an array for reducing a volume of the flow channel.

In a preferred embodiment, intervals between the press-fit points are configured to be set according to a part of the human body corresponding to the flow channel. The larger the intervals, the lower a flow resistance of the liquid caused by the press-fit points, and the smaller the intervals, the higher the flow resistance of the liquid caused by the press-fit points.

In a preferred embodiment, the inflatable layer comprises a plurality of independent airbags, and each of the plurality of independent airbags respectively comprises an air hole for air intake and air exhaust.

The present disclosure further provides a cold-hot compress device, comprising one or more of the carriers, and the main unit.

Compared with the existing techniques, the technical solution has the following advantages.

1. The present disclosure provides the carrier used in the cold-hot compress device. The cold-hot compress is applied to the human body by outputting the fluid from the main unit. With the carrier that has a large area and strong wrapping properties, a larger area of the cold-hot compress can be achieved. Moreover, since the carrier carries the fluid, a shape of the carrier can be bent at will, which can better fit physiological curves of the human body and thus achieve a better wrapping effect.

2. The present disclosure provides the carrier used in the cold-hot compress device. The one or more TPU layers are laminated on the inner side of fabric of the working layer. Tensile deformation resistance of the one or more TPU layers can reduce a defect of the working layer deforming after long-term use, which leads to a gradual increase in liquid consumption.

3. The present disclosure provides the carrier used in the cold-hot compress device. The flow channel disposed in the winding arrangement is defined in the working layer, which can increase a length of the flow channel, thereby increasing a contact time between the liquid and the human body when the liquid flows in the flow channel.

4. The present disclosure provides the carrier used in the cold-hot compress device, which reduces a volume of the flow channel and thus an amount of the liquid used by providing the press-fit points in the flow channel.

### BRIEF DESCRIPTION OF THE DRAWINGS

FIG. 1 is a schematic diagram of a carrier in a preferred embodiment of the present disclosure.

FIG. 2 is a schematic diagram of an internal structure of the carrier in the preferred embodiment of the present disclosure.

FIG. 3 is a schematic diagram of the carrier wrapped around a human body in the preferred embodiment of the present disclosure.

FIG. 4 is a layered structure diagram of the carrier in the preferred embodiment of the present disclosure.

FIG. 5 is a layered structure diagram of a working layer in the carrier in the preferred embodiment of the present disclosure.

FIGS. 6-11 are schematic diagrams of the carrier wrapped around different parts of the human body in the preferred embodiment of the present disclosure.

FIG. 12 is a schematic diagram of the carrier in an unfolded state used to wrap an arm in the preferred embodiment of the present disclosure;

FIG. 13 is a schematic diagram of internal flow channels of the carrier used to wrap the arm in the preferred embodiment of the present disclosure.

FIG. 14 is a schematic diagram of the carrier in an unfolded state used to wrap an upper body in the preferred embodiment of the present disclosure.

FIG. 15 is a schematic diagram of internal flow channels of the carrier used to wrap the upper body in the preferred embodiment of the present disclosure.

FIG. 16 is a schematic diagram of the carrier in an unfolded state used to wrap a waist in the preferred embodiment of the present disclosure.

FIG. 17 is a schematic diagram of internal flow channels of the carrier used to wrap the waist in the preferred embodiment of the present disclosure.

FIG. 18 is a schematic diagram of the carrier in an unfolded state used to wrap a single leg in the preferred embodiment of the present disclosure.

FIG. 19 is a schematic diagram of internal flow channels of the carrier used to wrap the single leg in the preferred embodiment of the present disclosure.

FIG. 20 is a schematic diagram of the carrier wrapping two legs respectively in the preferred embodiment of the present disclosure.

FIG. 21 is a schematic diagram of internal flow channels of the carrier that respectively wraps the two legs in the preferred embodiment of the present disclosure.

FIG. 22 is a schematic diagram of the carrier in an unfolded state that simultaneously wraps the two legs in the preferred embodiment of the present disclosure.

FIG. 23 is a schematic diagram of internal flow channels of the carrier that simultaneously wraps the two legs in the preferred embodiment of the present disclosure.

FIG. 24 is a schematic diagram of an inflated layer in the carrier used to wrap the arm in the preferred embodiment of the present disclosure.

FIG. 25 is an exploded diagram of the inflated layer in the carrier used to wrap the arm in the preferred embodiment of the present disclosure.

FIG. 26 is a schematic diagram of the inflated layer in the carrier used to wrap the upper body in the preferred embodiment of the present disclosure;

FIG. 27 is a schematic diagram of the inflated layer in the carrier used to wrap the waist in the preferred embodiment of the present disclosure.

FIG. 28 is a first schematic diagram of a main unit in the preferred embodiment of the present disclosure.

FIG. 29 is a second schematic diagram of the main unit in the preferred embodiment of the present disclosure.

FIG. 30 is a third schematic diagram of the main unit in the preferred embodiment of the present disclosure.

FIG. 31 is a layered structure diagram of the carrier in an alternative arrangement of the present disclosure.

FIG. 32 is a schematic diagram of the carrier wrapped around the human body in the alternative arrangement of the present disclosure.

### DETAILED DESCRIPTION OF THE EMBODIMENTS

In order to make the technical solution and the characteristics of the present disclosure clearer, the present disclosure will be further described in detail below with reference to the accompanying drawings and specific embodiments. It should be understood that these embodiments are merely used to illustrate the present disclosure and are not used to limit the scope of the present disclosure. Various equivalent modifications of the present disclosure fall within the scope defined by the appended claims of the present application provided that they are made by those of skill in the art after reading the present disclosure.

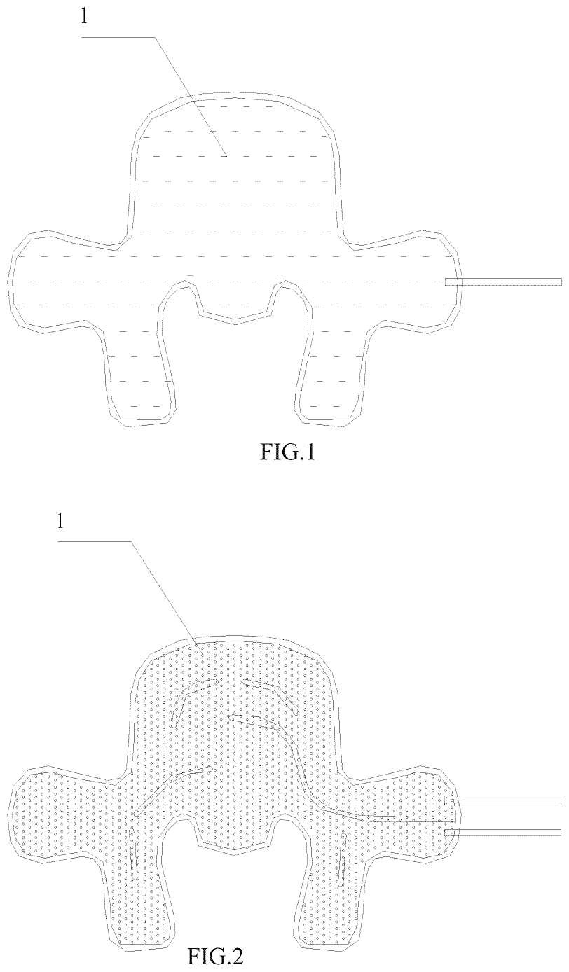

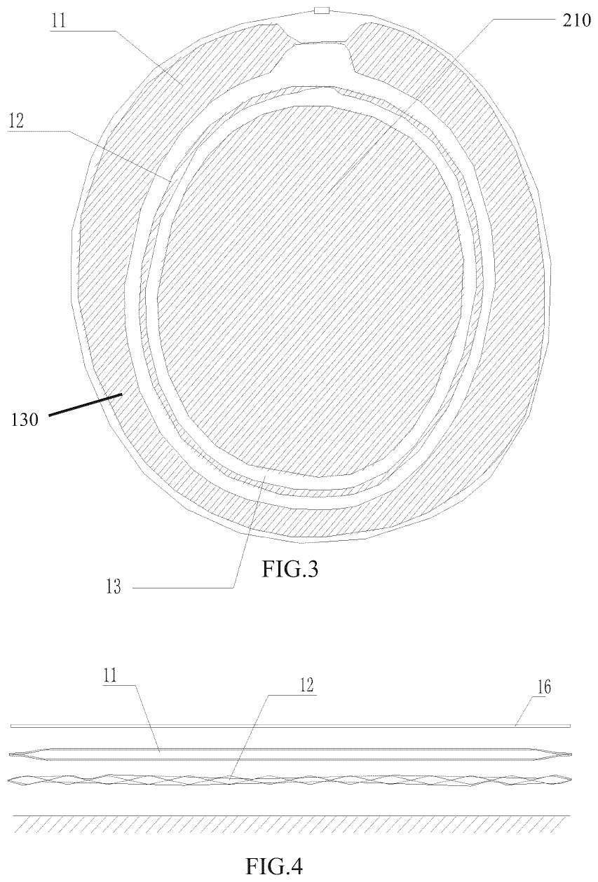

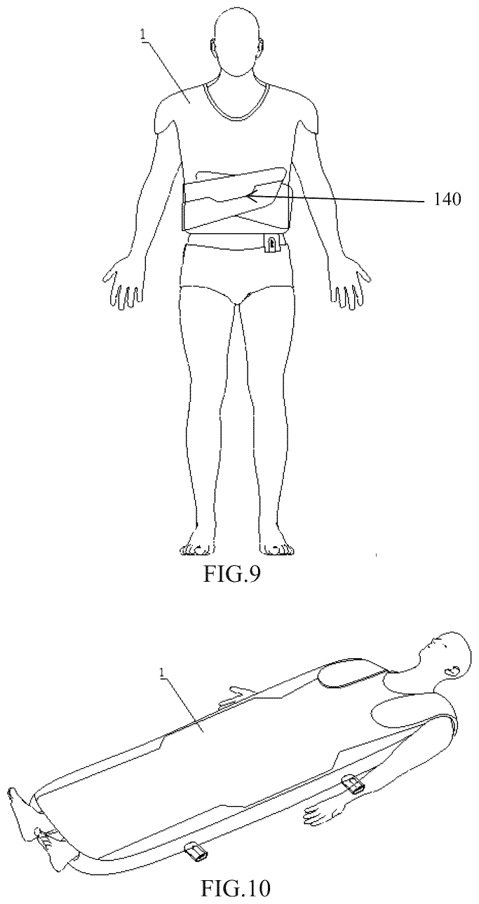

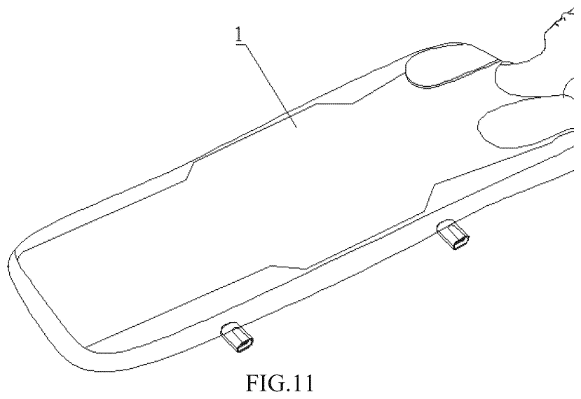

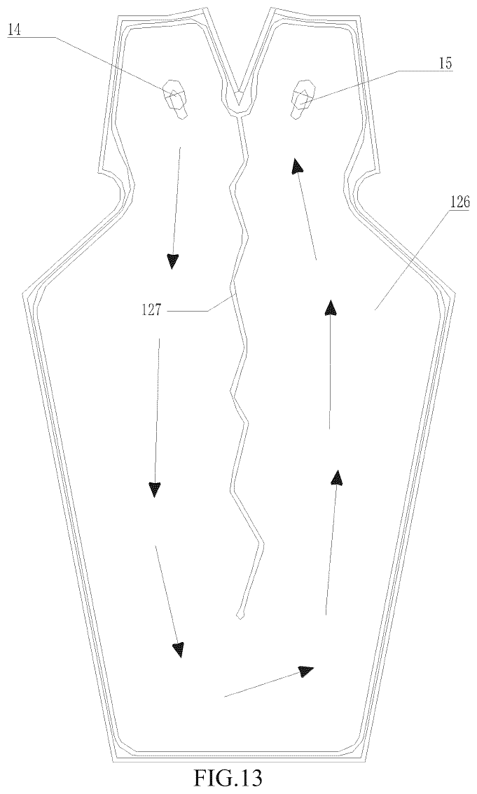

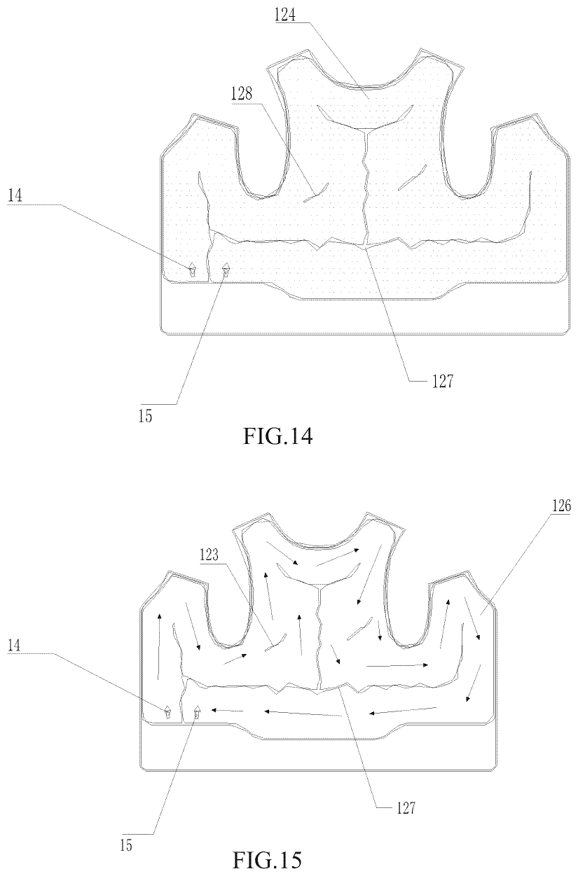

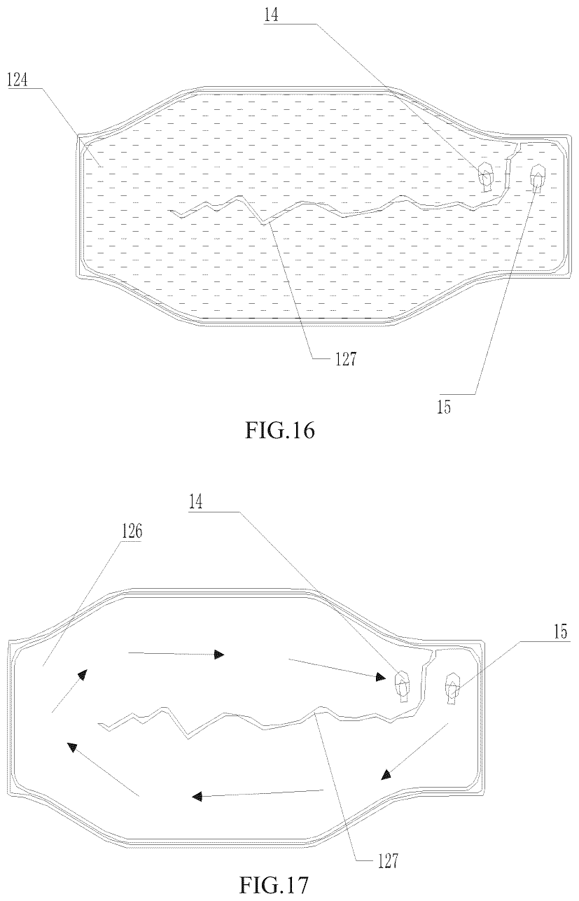

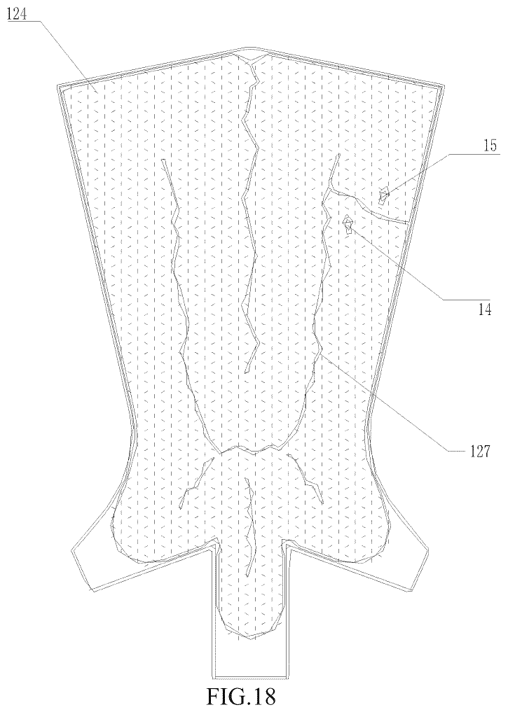

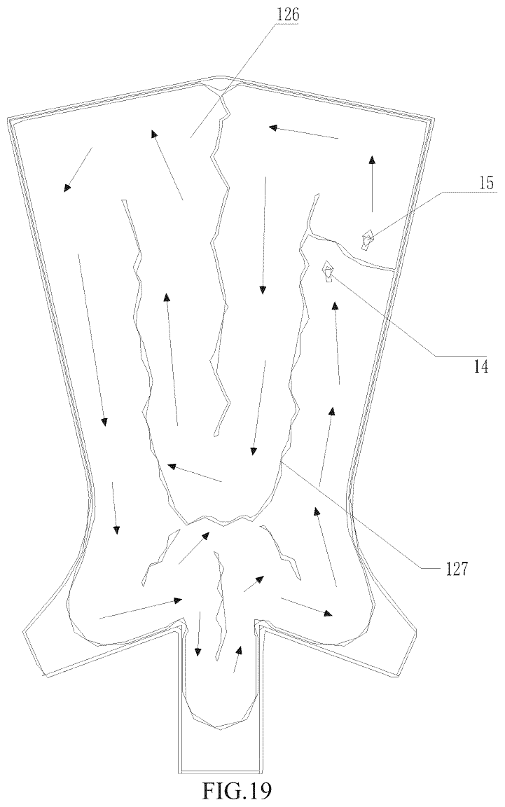

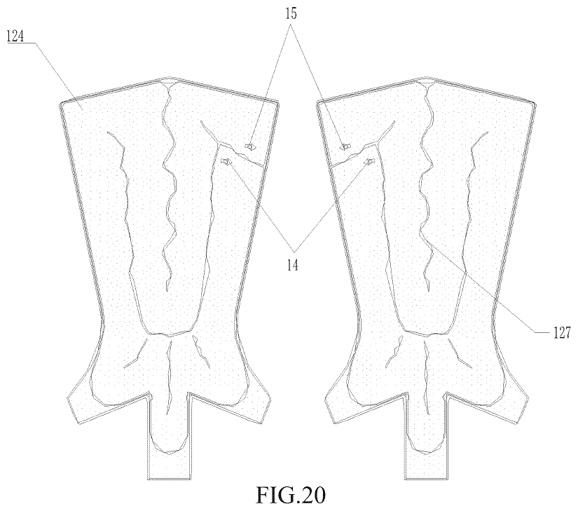

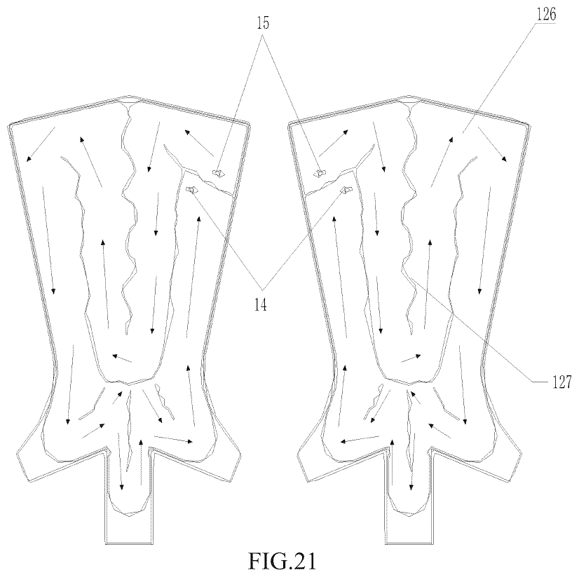

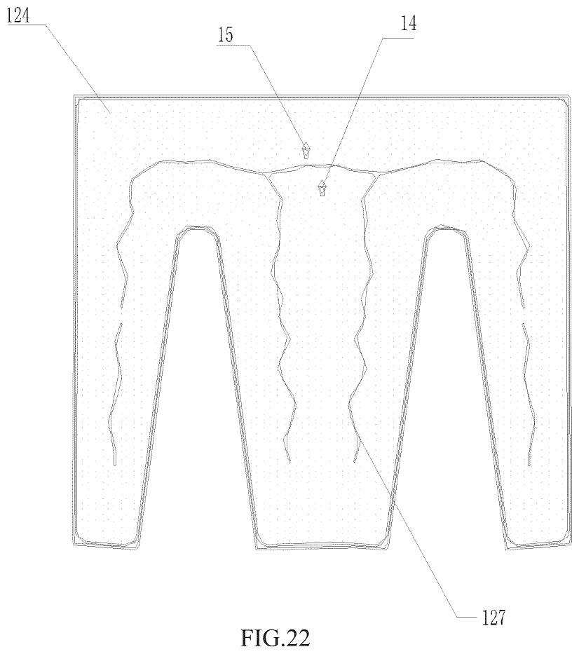

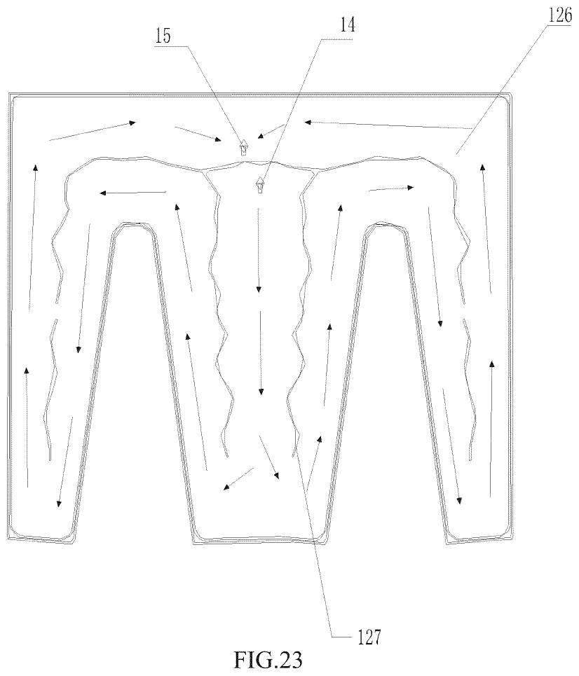

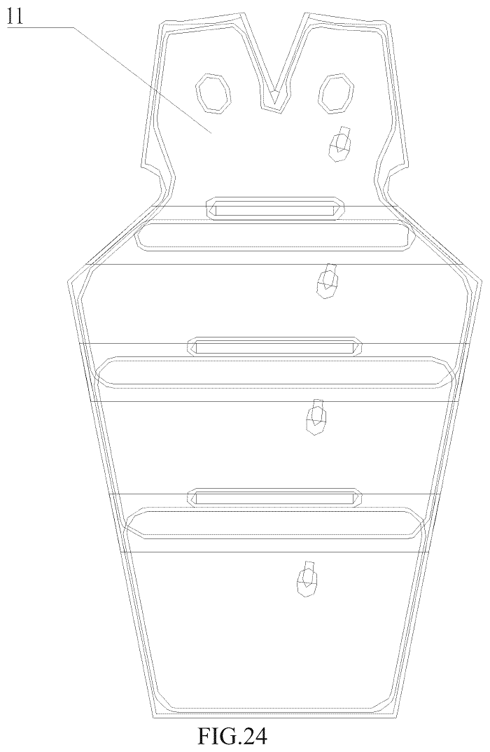

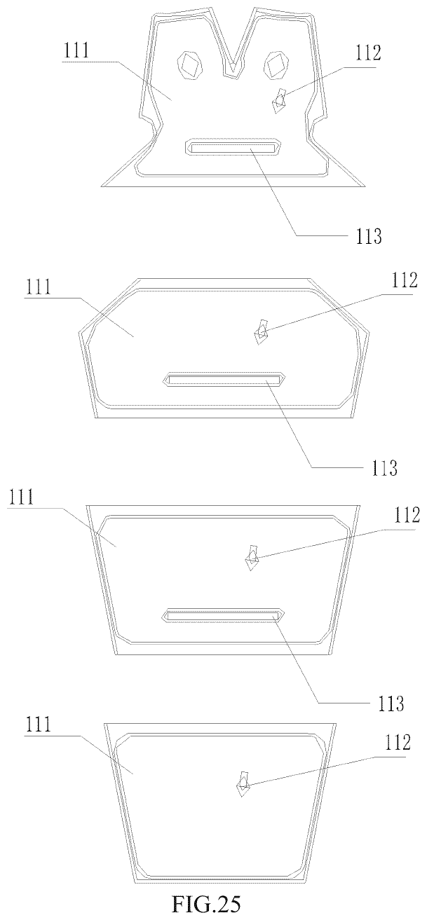

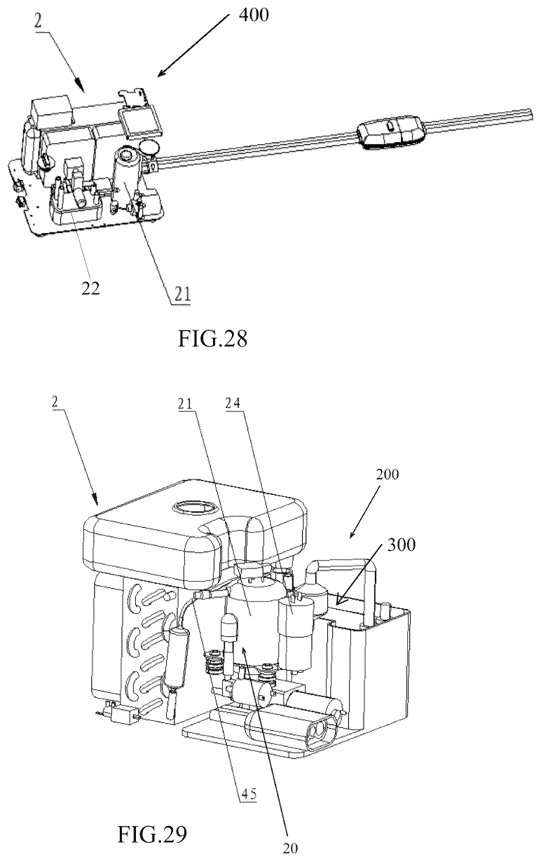

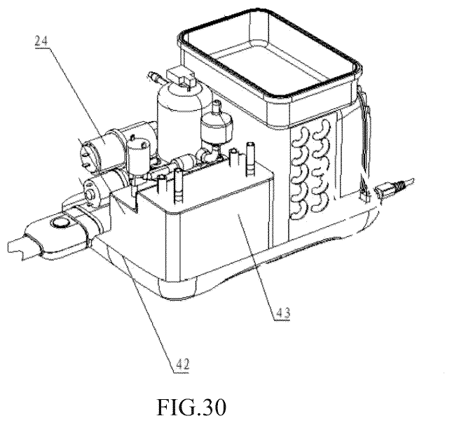

Referring to FIGS. 1-25 and FIGS. 28-30, a cold-hot compress device comprises one or more carriers 1 and a main unit 2. A cold-hot component 20 and a circulation component 200 are disposed in the main unit 2. The cold-hot component 20 comprises a compressor 21, a condenser 22, a heat exchanger 43, and a flow-restricting element 45, and the compressor 21, the condenser 22, the heat exchanger 43, and the flow-restricting element 45 are connected in sequence to achieve a cooling function. The cold-hot component 20 further comprises a heating element 42 (e.g., a heating plate or an electric heating wire) to achieve a heating function. The circulation component 200 comprises a storage tank 300, a pumping device 24, and a delivery pipe 400. The pumping device 24 is connected to the storage tank 300 and the one or more carriers 1 via the delivery pipe 400, and the delivery pipe 400 exchanges heat with the cold-hot component 20. The cold-hot component 20 is configured to deliver a fluid to the one or more carriers 1 or receive the fluid from the one or more carriers 1.

Through an arrangement of the circulation component 200, a medium (i.e., the fluid) is pumped by the pumping device 24 from the storage tank 300, exchanges heat with the cold-hot component 20, is then input into the one or more carriers 1 to exchange heat with a nursing part, and finally returns to the storage tank 300. A temperature of the medium in the delivery pipe 400 is adjusted through the cooling function and the heating function of the cold-hot component 20, so that a cold-hot compress function can be achieved after the user wears the one or more carriers 1. Compared with the traditional semiconductor cooling and heating, the cold-hot component 20 comprising a cooling part having the compressor 21, the condenser 22, the heat exchanger 43, and the flow-restricting element 45 and a heating part having the heating element 42 has a wider application range and a larger load and can be applied to cold-hot compress operations where a large area of the user is covered and with a high power.

Since the one or more carriers 1 carry the fluid, a shape of the one or more carriers 1 can be bent at will, better conforming to physiological curves of a human body 210, thus achieving a better wrapping effect. To this end, in this embodiment, each of the one or more carriers 1 comprises a main body 130 configured to be used for integrally wrapping a leg, a waist, an arm, an upper body, or a whole body. Therefore, this allows for a larger, snugger fitting effect, thus enhancing an effect of hot-and-cold compress.

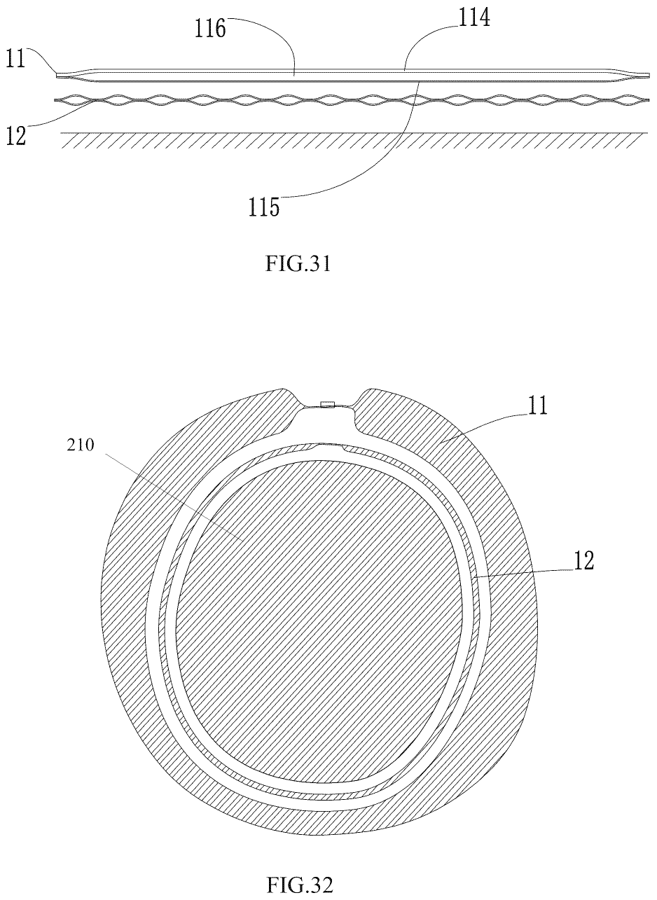

In this embodiment, to further enhance a fitting effect of the one or more carriers 1 onto various parts of the human body 210 during a cold-hot compressing process, the one or more carriers 1 can be inflated during the cold-hot compressing process to make the one or more carriers 1 fit skin of the human body 210 more closely. Therefore, the fluid output by the main unit 2 comprises air and liquid. Correspondingly, the main body 130 comprises an inflatable layer 11 for receiving the air and a working layer 12 for receiving the liquid, which are disposed in the main body 130. Specifically, the main body 130 of the carrier 1 comprises an outer layer 16, and the inflatable layer 11 and the working layer 12 for receiving the liquid are disposed on a side of the outer layer 16 facing the skin of the human body 210. As an alternative, the inflatable layer 11 can be omitted, and only the working layer 12 can be retained. Correspondingly, the fluid transported within the one or more carriers 1 is only the liquid. Referring to FIGS. 31-32, as another alternative, the outer layer 16 can be omitted. Specifically, the inflatable layer 11 comprises an outer fabric layer 114 and an inner thermoplastic polyurethane (TPU) layer 115, and the inner TPU layer 115 is attached with the outer fabric layer 114 to define an inflatable space 116 to receive the air. As a result, the outer fabric layer 114 of the inflatable layer 11 can serve a same function as the outer layer 16.

As the working layer 12 of the cold-hot compress device plays a role in cold-hot compress and the inflatable layer 11 only assists the working layer 12 in enabling the working layer 12 to fit more closely with the skin of the human body 210 to achieve a better effect, the inflatable layer 11 and the working layer 12 are stacked along a thickness direction of a cavity 13 of the main body 130, and the working layer 12 is disposed on an inner side of the inflatable layer 11. In this way, the working layer 12 can directly contact the skin of the human body 210, and cold-hot compress effects will not decrease due to an arrangement of the inflatable layer 11. The inflatable layer 11 is disposed on an outer side of the working layer 12, and when the inflatable layer 11 expands, the working layer 12 can approach in a direction close to the skin of the human body 210 under extrusion from the inflatable layer 11, so as to achieve a better fitting effect.

In addition, during long-term use, a surface fabric of the working layer 12 is stretched and deformed due to repeated filling and discharging of the liquid, resulting in an increase in a volume of the working layer 12 and an increase in a used amount of the liquid. The increase in the used amount of the liquid will lead to a decrease in an efficiency of the cold-hot compress. In order to solve this problem, fabrics of the working layer 12 are divided into one or more cloth base layers 121 and one or more thermoplastic polyurethane (TPU) layers 122, and the one or more TPU layers 122 are disposed on one or more inner sides of the one or more cloth base layers 121. Moreover, the one or more TPU layers 122 in an upper fabric and a lower fabric of the working layer 12 are connected at a certain distance by pressing along an extending direction of the working layer 12 to form a connection section 125. That is, the one or more TPU layers 122 in the upper fabric and the lower fabric of the working layer 12 are connected to define the volume of the working layer 12. TPU material of the one or more TPU layers 122 has good tensile and deformation resistance. Therefore, during a liquid filling and discharging process of the working layer 12, even if the one or more cloth base layers 121 are stretched and deformed, the volume of the working layer 12 can be maintained at a relatively stable level due to a performance of the tensile and deformation resistance of the one or more TPU layers 122. Thus, a situation in which the volume of the working layer 12 increases and the amount of the liquid used increases will not occur after the long-term use.

In this embodiment, the main body 130 has the cavity 13 for integrally wrapping the leg, the waist, the arm, the upper body, or the whole body, and a shape of the cavity 13 is designed according to a shape of different parts of the human body 210. Specifically, since the arm of the human body 210 has a curve that is wide at an upper part and narrow at a lower part, when a corresponding one of the one or more carriers 1 for wrapping the arm of the human body 210 is unfolded into a flat state, a width of the corresponding one of the one or more carriers 1 has a structure that gradually decreases from a middle to a lower end, so that the cavity 13 corresponding to the shape of the arm can be formed when the corresponding one of the one or more carriers 1 is wrapped around the arm of the human body 210 by winding. Moreover, a connection structure 140 is disposed on the main body 130 for changing the one or more carriers 1 between an unfolded state and a wrapped state. Specifically, the connection structure 140 can adopt a quick disassembly and assembly design such as a zipper structure or a hook and loop fastener structure. It should be noted that a corresponding one of the one or more carriers 1 for wrapping the leg can be divided into a single-leg carrier 1 for wrapping a single leg and a two-leg carrier 1 for wrapping two legs, wherein the two-leg carrier 1 for wrapping the two legs can wrap the two legs simultaneously in a form of trousers or can wrap the two legs respectively in two independent sub-carriers. It should be noted that the cavity 13 can also be directly formed on the one or more carriers 1, rather than the cavity 13 being formed when the one or more carriers 1 are wrapped around the human body 210. For example, the corresponding one of the one or more carriers 1 directly has two of the cavities 13 for wrapping the two legs when being in the form of the trousers.

A specific structure of the working layer 12 will be described below. In order to achieve liquid circulation, a liquid inlet 14 and a liquid outlet 15 that are in communication with the working layer 12 are further disposed on the main body 130. The liquid flows into the working layer 12 through the liquid inlet 14 from the main unit 2 and then flows back to the main unit 2 from the liquid outlet 15 to achieve the liquid circulation. In order to avoid a situation in which a flow path of the liquid between the liquid inlet 14 and the liquid outlet 15 is too short, resulting in the liquid failing to pass through the entire working layer 12, a flow channel 126 that the liquid flows through needs to be increased as much as possible, and a contact time and a contact area between the liquid and the skin of the human body 210 can be increased, thereby improving an effect of the cold-hot compress. For this purpose, a first indentation 127 is formed in the working layer 12. The first indentation 127 is formed by connecting the one or more TPU layers 122 in the upper fabric and the lower fabric of the working layer 12 by pressing, so that the flow channel 126 in communication with the liquid inlet 14 and the liquid outlet 15 is defined in the working layer 12 by the first indentation 127. The flow channel 126 is disposed in a winding arrangement. The phrase “winding arrangement” means that the flow channel 126 comprises at least one corner, and the liquid turns by a certain angle when flowing through the at least one corner. A number of the at least one corner can be set according to different parts of the body. For example, for corresponding ones of the one or more carriers 1 for wrapping the arm and the waist, the number of the at least one corner can be set to 1. For corresponding ones of the one or more carriers 1 for wrapping the upper body or the leg, the number of the at least one corner needs to be increased.

In addition, for some specific applications, for example, a width of the flow channel 126 of a corresponding one of the one or more carriers 1 for wrapping the upper body in a position of a chest of the upper body is relatively wide, and the liquid may be uneven locally when flowing through this part. That is, the liquid is more in some sections and less in other sections. Alternatively a flow rate of the liquid in one section may be too fast, resulting in a contact time between the liquid and the skin of the human body 210 being too short. In this case, a second indentation 123 is further formed in the working layer 12, and a certain distance is formed between the second indentation 123 and the first indentation 127 at a position where the second indentation 123 is located, so as to divide the liquid flowing through the second indentation 123 into two parts. In this way, the liquid can flow out from both sides of the second indentation 123, and the second indentation 123 can block the liquid at the same time, so that the liquid is more uniform when passing through the one section. The contact time with the skin of the human body 210 can be increased accordingly. A main purpose of arranging the first indentation 127 and the second indentation 123 is to allow the liquid to flow through all sections in the working layer 12 of the corresponding one of the one or more carriers 1. Whether the first indentation 127 is present depends on a width of the working layer 12. If the width of the working layer 12 is relatively wide and a flow amount of the liquid flowing into the working layer 12 is limited, the limited liquid cannot fill the entire working layer 12. At this time, the first indentation 127 and the second indentation 123 are needed to divide the working layer 12 into winding flow channels to reduce the width of the flow channel 126. Thus, the liquid can flow through the largest possible section. If the inherent width of the working layer 12 is relatively small, it is not necessary to have the first indentation 127 and the second indentation 123.

In addition, in order to reduce a used amount of the liquid, so as to achieve a rapid temperature rise and temperature fall of the one or more carriers 1, press-fit points 124 for reducing a volume of the flow channel 126 are disposed in an array in the working layer 12. As the press-fit points 124 occupy a part of a space in the flow channel 126, the volume in the flow channel 126 is reduced, and the used amount of liquid can be reduced naturally. Intervals between the press-fit points 124 and sizes of the press-fit points 124 can be set according to different parts of the human body 210 corresponding to the flow channel 126. The larger the intervals, the lower a flow resistance of the liquid caused by the press-fit points 124, and the lower the flow rate of the liquid flowing through a part of the different parts. Conversely, the smaller the intervals, the higher the flow resistance of the liquid caused by the press-fit points 124, and the higher the flow rate of the liquid flowing through the part of the different parts. Therefore, sizes of the intervals can be set corresponding to actual needs to obtain the best effect. It should be noted that the sizes of the intervals between the press-fit points 124 and the sizes of the press-fit points 124 can be set according to the different parts of the human body 210 corresponding to the flow channel 126, which means that the sizes of the intervals between the press-fit points 124 can be different in different carriers 1 or the sizes of the intervals between the press-fit points 124 can also be different in different locations within the same carrier 1. For example, in the carrier 1 for wrapping two legs, the sizes of the intervals between the press-fit points 124 are different in different sections.

A structure of the inflatable layer 11 will be described below. The inflatable layer 11 comprises a plurality of independent airbags 111, and each of the plurality of independent airbags 111 respectively comprises an air hole 112 for air intake and air exhaust. Both of the air intake and the air exhaust can be achieved through a same air hole 112, and the pumping device 24 switches working modes to achieve the air intake or the air exhaust through the air hole 112, so as to achieve air inflation and deflation processes of the plurality of independent airbags 111. The inflatable layer 11 is divided into the plurality of independent airbags 111 so as to accelerate the air inflation and deflation speed of each of the plurality of independent airbags 111, and at the same time, a certain airbag or certain several airbags 111 of the plurality of independent airbags 111 can be inflated and deflated independently according to different mode selections. Alternatively, the plurality of airbags 111 can be inflated and deflated according to a certain rule to achieve a massage effect. In addition, each of the plurality of independent airbags 111 has a section 113 combined with another of the plurality of independent airbags 111. Two adjacent ones of the plurality of independent airbags 111 are joined via the section 113 to form the complete inflatable layer 11.

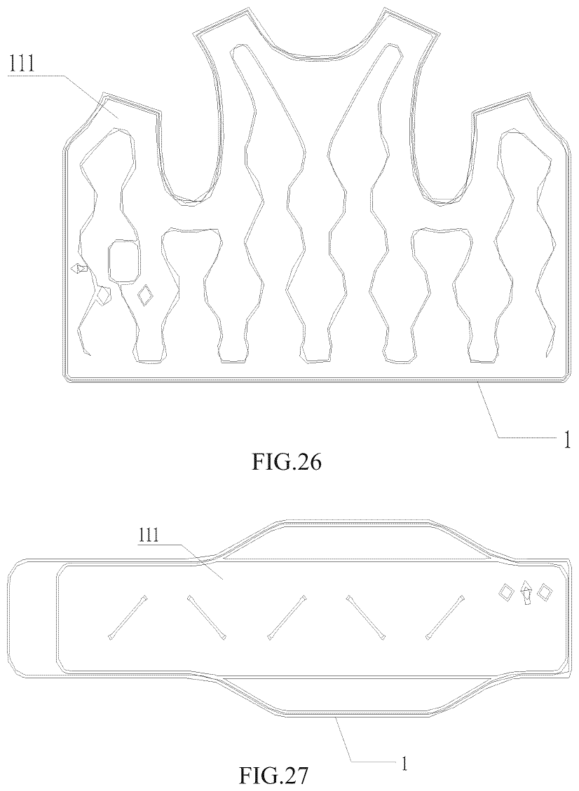

Not all of the inflatable layer 11 of the carrier 1 comprises the plurality of independent airbags 111, and the inflatable layer 11 of each of the one or more carriers 1 may comprise only one of the plurality of independent airbags 111. For example, corresponding ones of the one or more carriers 1 for wrapping the upper body or the waist only has one of the plurality of independent airbags 111, as shown in FIGS. 26 and 27.

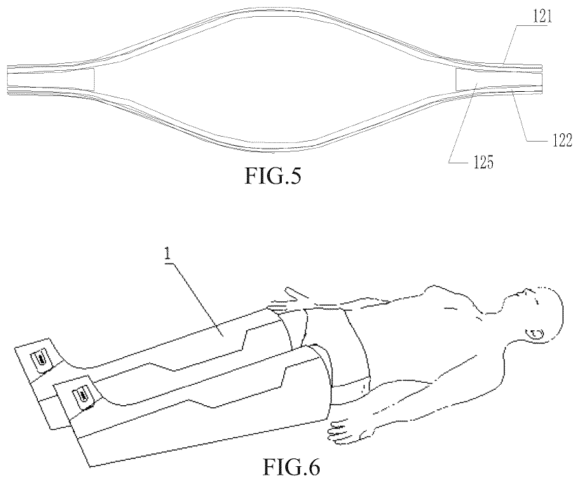

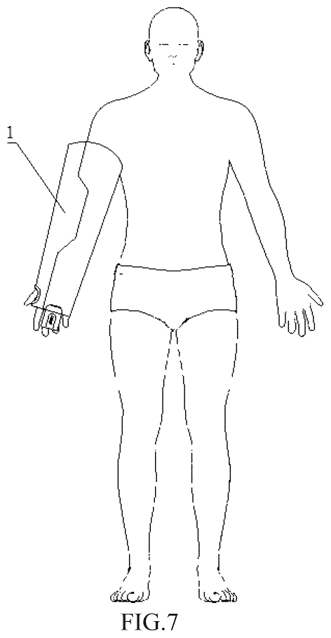

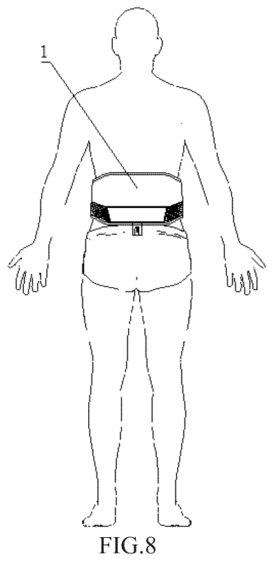

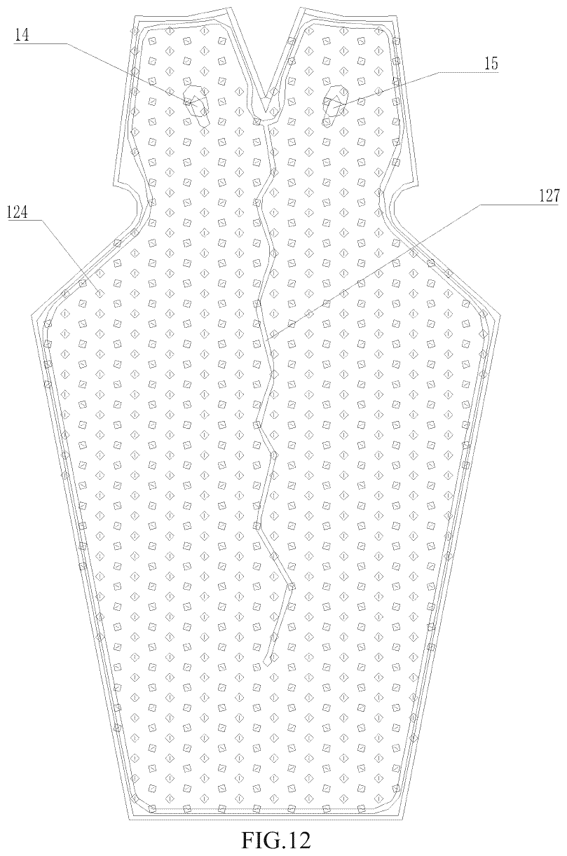

FIGS. 6-11 are only used to illustrate parts of the human body 210 that are possible to be wrapped using the one or more carriers 1. The one or more carriers 1 in FIGS. 6-11 are simplified, so the configurations of the one or more carriers 1 in FIGS. 6-11 are different from the various specific styles of the carrier 1 in FIGS. 12-23.

The preceding description is merely a specific embodiment of the present disclosure, but the design and the concept of the present disclosure is not limited thereto. It is intended that the protective scope of the present disclosure cover any non-substantial modification provided it is made using the concept.

What is claimed is:

1. A carrier used in a cold-hot compress device, wherein:
the carrier is configured to be connected to a main unit of the cold-hot compress device so as to receive a fluid output by the main unit, and
the carrier comprises a main body configured to be used for integrally wrapping one or more legs, a waist, an arm, an upper body, or a whole body of a human body.

2. The carrier used in the cold-hot compress device according to claim 1, wherein:
the fluid comprises air and liquid.

3. The carrier used in the cold-hot compress device according to claim 2, wherein:
the main body comprises an outer layer, an inflatable layer, and a working layer,
the inflatable layer and the working layer are disposed on a side of the outer layer facing skin of the human body,
the inflatable layer is configured to receive the air output by the main unit, and
the working layer is configured to receive the liquid output by the main unit.

4. The carrier used in the cold-hot compress device according to claim 3, wherein:
the inflatable layer and the working layer are stacked along a direction perpendicular to the skin of the human body.

5. The carrier used in the cold-hot compress device according to claim 1, wherein:
the fluid is a liquid,
the main body comprises an outer layer and a working layer,
the working layer is disposed on a side of the outer layer facing skin of the human body, and
the working layer is configured to receive the liquid output by the main unit.

6. The carrier used in the cold-hot compress device according to claim 3, wherein:
fabrics of the working layer are divided into one or more cloth base layers and one or more thermoplastic polyurethane (TPU) layers, and
the one or more TPU layers in an upper fabric and a lower fabric of the working layer are connected at a certain distance by pressing along an extending direction of the working layer.

7. The carrier used in the cold-hot compress device according to claim 1, wherein:
the main body comprises one or more cavities that integrally wrap the one or more legs, the waist, the arm, the upper body, or the whole body of the human body, and
a shape of the one or more cavities is set according to a shape of a part of the human body being wrapped.

8. The carrier used in the cold-hot compress device according to claim 3, wherein:
the main body comprises a liquid inlet and a liquid outlet in communication with the working layer.

9. The carrier used in the cold-hot compress device according to claim 8, wherein:
a first indentation is provided in the working layer,
the first indentation is configured to define a flow channel in the working layer that connects the liquid inlet to the liquid outlet, and
the flow channel is disposed in a winding arrangement.

10. The carrier used in the cold-hot compress device according to claim 9, wherein:
a second indentation is further provided in the working layer, and
a certain distance is formed between the second indentation and the first indentation at a position where the second indentation is located, so as to divide the liquid flowing through the second indentation into two parts.

11. The carrier used in the cold-hot compress device according to claim 10, wherein:
the first indentation and the second indentation are curved pressing lines.

12. The carrier used in the cold-hot compress device according to claim 9, wherein:
the working layer is provided with press-fit points in an array for reducing a volume of the flow channel.

13. The carrier used in the cold-hot compress device according to claim 12, wherein:
intervals between the press-fit points are configured to be set according to a part of the human body corresponding to the flow channel,
the larger the intervals, the lower a flow resistance of the liquid caused by the press-fit points, and
the smaller the intervals, the higher the flow resistance of the liquid caused by the press-fit points.

14. The carrier used in the cold-hot compress device according to claim 3, wherein:
the inflatable layer comprises a plurality of independent airbags, and
each of the plurality of independent airbags respectively comprises an air hole for air intake and air exhaust.

15. A cold-hot compress device, comprising:
one or more of the carriers according to claim 1, and
the main unit.

16. The carrier used in the cold-hot compress device according to claim 5, wherein:
fabrics of the working layer are divided into one or more cloth base layers and one or more thermoplastic polyurethane (TPU) layers, and
the one or more TPU layers in an upper fabric and a lower fabric of the working layer are connected at a certain distance by pressing along an extending direction of the working layer.

17. The carrier used in the cold-hot compress device according to claim 5, wherein:
the main body comprises a liquid inlet and a liquid outlet in communication with the working layer.

18. The carrier used in the cold-hot compress device according to claim 17, wherein:
a first indentation is provided in the working layer,
the first indentation is configured to define a flow channel in the working layer that connects the liquid inlet to the liquid outlet, and
the flow channel is disposed in a winding arrangement.

19. The carrier used in the cold-hot compress device according to claim 18, wherein:
a second indentation is further provided in the working layer, and
a certain distance is formed between the second indentation and the first indentation at a position where the second indentation is located, so as to divide the liquid flowing through the second indentation into two parts.

20. The carrier used in the cold-hot compress device according to claim 1, wherein:
the main body comprises an inflatable layer and a working layer,
the fluid comprises air and liquid,
the inflatable layer is configured to receive the air output by the main unit, and
the working layer is configured to receive the liquid output by the main unit.
